(19) United States
(12) Patent Application Publication
(10) Pub. No.: US 2003/0039618 A1
(43) Pub. Date: Feb. 27, 2003

(54) Persicae flos extract-containing composition for skin protection against UV

(75) Inventors: Kang Tae Lee, Chungcheongnam-do (KR); Byoung Kee Jo, Kyunggi-do (KR); Hyun Pyo Kim, Seoul (KR); Moon Young Heo, Kangwon-do (KR)

Correspondence Address: FINNEGAN, HENDERSON, FARABOW, GARRETT &, DUNNER LLP, 1300 I STREET, NW, WASHINGTON, DC, 20005, US

(73) Assignee: Coreana Cosmetics Co.,

(21) Appl. No.: 10/146,390

(22) Filed: May 16, 2002

Related U.S. Application Data
(62) Division of application No. 09/819,656, filed on Mar. 29, 2001.

(30) Foreign Application Priority Data
Aug. 9, 2000 (KR) 2000-46178

(51) Int. Cl. A61K 35/78; A61K 7/42

(52) U.S. Cl. 424/59; 424/765

(57) ABSTRACT

The present invention relates to a Persicae Flos extract-containing composition for skin-protection against UV.

The Persicae Flos extract according to the present invention shows a long-term effect against skin aging and skin cancer, by absorbing UV light without exhibiting skin irritation and, at the same time by inhibiting undesirable activation of skin cells due to UV exposure.

### FIELD OF THE INVENTION

The present invention relates to a Persicae Flos extract-containing composition for skin protection against UV.

### BACKGROUND OF THE INVENTION

It was already established through various experiments on human skin that UV light has a negative effect on skin.

When exposed to UV light, keratinocyte among skin cells secretes cytokine that causes oxidative stress, leading to activation of various types of proteins within the cell. These activated enzymes hydrolyze cell membrane lipid, resulting in secretion of arachidonic acid. The secreted arachidonic acid is transformed into prostaglandin E₂ (PGE₂) by cyclooxygenase. Thus formed arachidonic acid and PGE₂, together with cytokine, cause inflammatory reactions such as skin ruber, which lead to stimulation of skin aging.

In addition, fibroblast among skin cells is also activated upon UV exposure by a procedure similar to that of the keratinocyte activation described above. Fibroblast may be destroyed as the amount of UV light increases, and skin aging is stimulated by the damage of fibroblast DNA, possibly leading to a skin cancer.

In other words, UV light causes skin ruber, inhibition of immunity, wrinkle formation and eventually skin aging and skin cancer. Therefore, preventing said phenomena occurred in skin cells by UV exposure is critical for the prevention of skin cancer and the retardation of aging.

Recently, increase of UV light and change of UV region due to the destruction of ozone layer stimulate skin aging and skin cancer, and still, further increase is expected.

Therefore, protecting human skin against UV light is very important in cosmetics industry. Currently, simple UV blocking agent or UV absorbing agent is used to prevent the damage by UV exposure. However, it has been reported that UV blocking agent itself may cause mutagenesis, leading to skin cancer.

### SUMMARY OF THE INVENTION

The present invention provides a phytophysiologically active substance which has a long-term effect against skin aging and skin cancer, not only by absorbing UV without skin irritation but also by basically inhibiting the undesirable activation of skin cells upon UV exposure.

### BRIEF DESCRIPTION OF THE DRAWINGS

FIG. 1 shows UV-A absorbance of Persicae Flos extract in Example 4.

FIG. 2 shows UV-B absorbance of Persicae Flos extract of Example 4.

FIG. 3 shows amount of arachidonic acid secreted from keratinocyte (CPM) when treated with 0, 100, 500 and 1000 μg/ml of Persicae Flos extract of Example 4, respectively, immediately before UV-B irradiation (n=3, *p<0.01).

FIG. 4 shows amount of arachidonic acid secreted from keratinocyte (CPM) when treated with 0, 100, 500 and 1000 μg/ml of Persicae Flos extract of Example 4, respectively, immediately after UV-B irradiation (n=3, *p<0.01).

FIG. 5 shows cell survival rate of keratinocyte when treated with 0, 100, 500 and 1000 μg/ml of Persicae Flos extract of Example 4, respectively, immediately before UV-B irradiation (n=3).

FIG. 6 shows amount of arachidonic acid and its metabolite, PGE₂, secreted from keratinocyte when treated with 500 μg/ml of Persicae Flos extract according to Example 4, immediately before UV-B exposure.

### DETAILED DESCRIPTION OF THE INVENTION

The present invention relates to a Persicae Flos extract-containing composition for skin-protection against UV.

Persicae Flos refers to a dried flower of peach (Prunus persicae L.), and it has been used for internal and external use. Chinese Pharmaceutical Dictionary (Sanghai Scientific Technologies Publishing Co., section of, 3rd vol., pp1923˜1924) discloses that Persicae Flos has been used internally for the treatment of constipation, spinal diseases and stomachache, and in particular, externally for the treatment of chronic eczema. However, there was no single report that external application of Persicae Flos or its extract may inhibit skin aging or skin cancer.

The Persicae Flos extract of present invention is prepared by conventional plant-extraction method. For example, dried Persicae Flos is extracted with organic solvent or a mixture of organic solvent and water, and the resultant is filtered and subjected to vacuum concentration to form extract.

The composition of the present invention comprises Persicae Flos extract in an amount of 0.01-10% by weight.

The composition of the present invention not only absorbs UV, but also inhibits UV activation of keratinocyte and fibroblast, thereby preventing harmful action due to UV exposure. Said effects can be accomplished by the application of the composition before UV irradiation, but unlike the conventional UV blocking agent, it can be also accomplished by the application immediately after UV irradiation.

To accomplish said effects, the Persicae Flos extract-containing composition according to the present invention can be applied to skin in an amount of 0.01-100 mg/cm² of Persicae Flos extract.

Examples of the formulations of the skin-protection composition of the present invention include, but are not limited to, skin softener, astringent, nutrient emulsion, eye cream, nutrient cream, massage cream, cleansing cream, cleansing foam, cleansing water, powder, essence and facial pack.

The present invention is more specifically explained by Examples and Experimental Example below, but not limited thereby.

### Examples 1˜4

### Preparation of Persicae Flos extract

100 g of Dried Persicae Flos was extracted with each 300 ml of organic solvent at ambient temperatures for 3 days. This was filtered and the filtrate was subjected to vacuum concentration to form extract. Yield (%) of the extract is given in Table 1.

Table 1. Yield of Extract Depending on Solvent (n=3)

| | Organic Solvent | Yield (%) |
|---|---|---|
| Example 1 | Methanol (100%) | 12.3 ± 2.2 |
| Example 2 | Aqueous solution of methanol (80%) | 21.1 ± 3.8 |
| Example 3 | Ethanol (100%) | 4.9 ± 2.0 |
| Example 4 | Aqueous solution of ethanol (80%) | 17.2 ± 3.3 |

### Example 5

### Persicae Flos-containing Composition

Among the compositions containing Persicae Flos extract of Example 4, the composition of cream type formulation is as follows.

| Component | Content (% by weight) |
|---|---|
| Persicae Flos extract in Example 4 | 3.0 |
| Meadow foam oil | 3.0 |
| Stearyl(?) alcohol | 1.5 |
| Stearic acid | 1.5 |
| Glyceryl stearate | 1.5 |
| Liquid paraffin | 10.0 |
| Bess wax | 2.0 |
| Polysorbate 60 | 2.5 |
| Sorbitan sesquioleate | 0.6 |
| Squalene | 3.0 |
| Lecithin | 2.5 |
| Corn oil | 4.0 |
| Oleic acid | 1.2 |
| 1,3-butylene glycol | 3.0 |
| glycerin | 5.0 |
| triethanol amine | 0.5 |
| tocopheryl acetate | 0.5 |
| preserving agent | 0.02 |
| flavor | 0.01 |
| purified water | q.s. |
| Total | 100 |

### Experimental Example 1

### UV absorption Activity

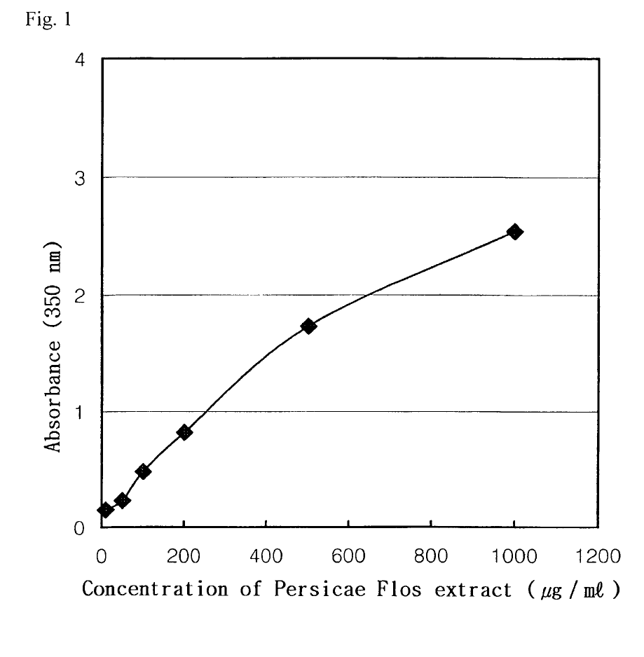

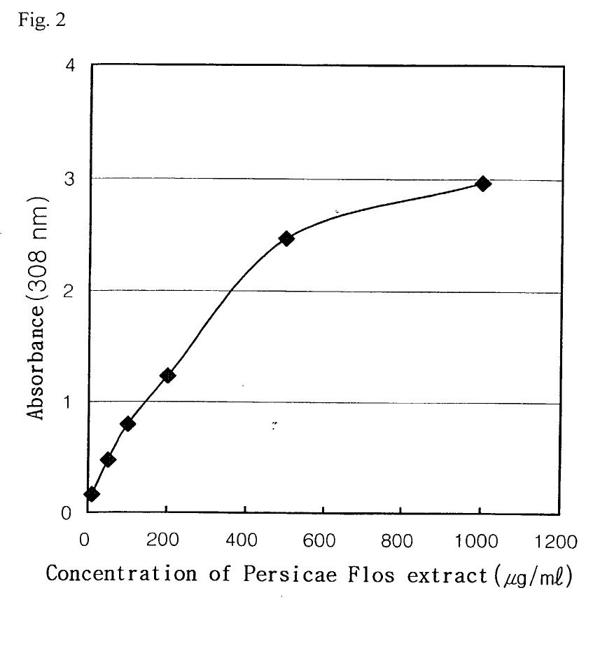

The Persicae Flos extract prepared in Example 4 was dissolved in water to be a concentration of 0.1, 1, 10, 100, 200, 500 and 1000 μg/ml, respectively, and each absorbance was measured at UV region (UV-A: 320-400 nm, UV-B: 280-320 nm, UV-C: 200-280 nm). The absorbance at Uv-A and UV-B is shown in FIGS. 1 and 2.

As can be seen in FIGS. 1 and 2, the Persicae Flos extract of the present invention shows strong absorbance at UV-A and UV-B regions, as well as UV-C region where most plant-extract exhibits high absorbance, and the UV absorbance is proportional to the concentration.

### Experimental Example 2

### Activation of Human Keratinocyte by UV

Upon radiation of UV light (UV-B) to keratinocyte, arachidonic acid is secreted therefrom; the secreted arachidonic acid is changed into prostaglandin E2 by the action of cyclooxygenase. Thus formed arachidonic acid and PGE₂, together with cytokines, cause many inflammatory reactions such as skin ruber, leading to skin aging. Therefore, the inhibitory effect of the Persicae Flos extract of the present invention on the activation of keratinocyte was evaluated by determining the amount of arachidonic acid secreted and PGE₂ from keratinocyte upon UV radiation, after the treatment with the Persicae Flos extract of the present invention.

### Experimental Example 2-1

Tissue obtained from children's circumcision was subjected to a trypsin treatment to obtain keratinocyte. Thus obtained keratinocyte was incubated in 24-well plate by using serum-free keratinocyte culture media (Gibco Company, USA). When cells showed 70-80% growth, they were treated with [3H] arachidonic acid (USA NEN company) in an amount of 0.0375 μCi/well and were incubated for 24 hours. Then, the culture was washed with new culture media twice, culture media was removed, 1 ml of phosphate buffer solution (PBS) was added, UV-B irradiation was carried out to 30 mJ/cm², culture media was again filled and incubated for 6 hours. Culture media was recovered, extracted with ethyl acetate, and the amount of secreted arachidonic acid and metabolite was determined by TLC (Thin Layer Chromatography) and radiation detector. At this time, 0, 100, 500 and 1000 μg/ml of the Persicae Flos extract obtained from the above Example 4 was added respectively to PBS immediately before UV irradiation or to culture media immediately after UV irradiation, and secreted arachidonic acid was assayed against control group without UV irradiation.

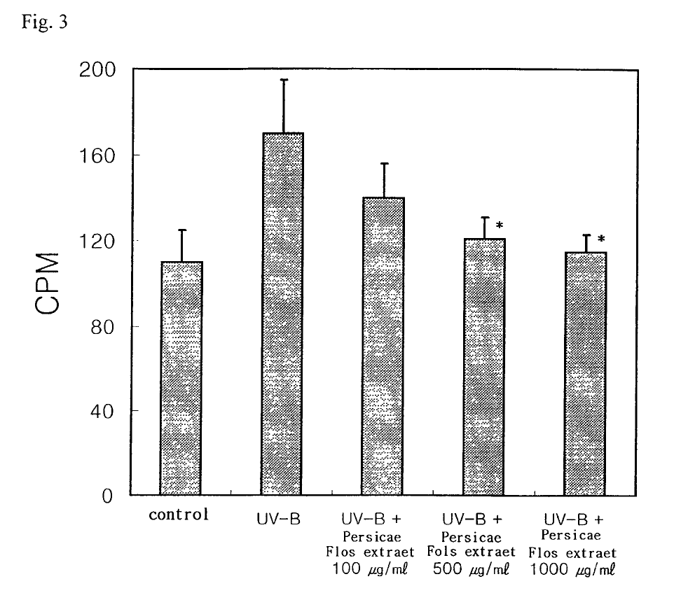

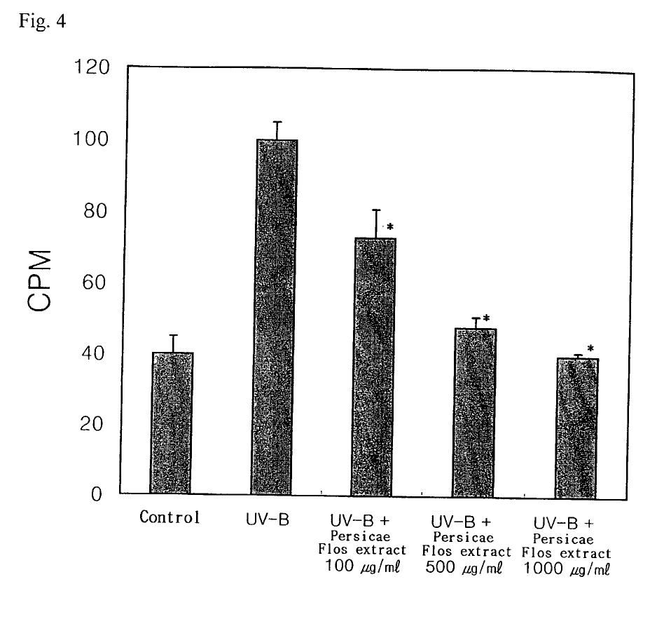

The results are shown in FIGS. 3 and 4. As can be seen in FIGS. 3 and 4, it was clearly revealed that the activation of keratinocyte by UV irradiation was inhibited by the Persicae Flos extract, regardless of whether the treatment was conducted before or after UV irradiation. The inhibitory activity was also proportional to the amount of the extract used.

### Experimental Example 2-2

Additionally, to confirm that the result of Experimental Example 2-1 was not resulted from error due to release of marker, an experiment identical with Experimental Example 2-1 was repeated using concentration of 500 μg/ml, UV-B was irradiated, cultivation for 6 hours was conducted, and so obtained ethyl acetate extract was subjected to following procedure.

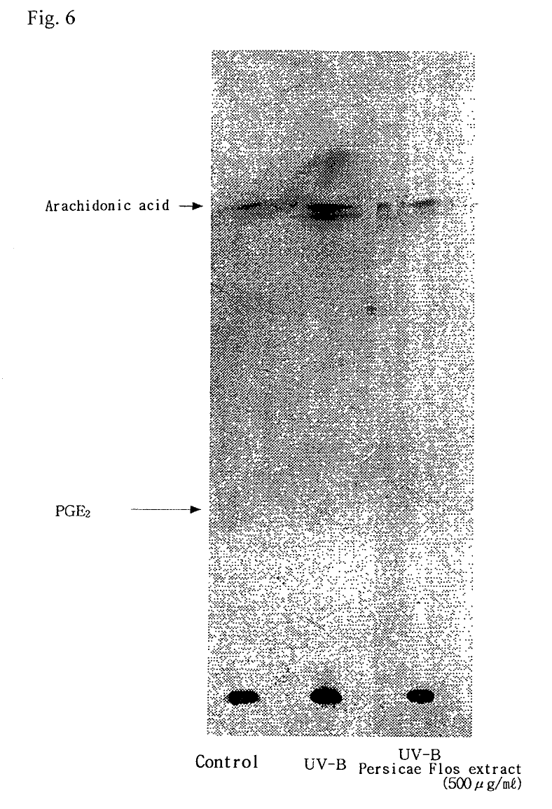

Lipid was extracted, from said extract, by Folch solution, separated by TLC, and arachidonic acid and its metabolite, PGE₂, were compared and identified by autoradiography. The result is shown in FIG. 6.

As seen in FIG. 6, when treated with Persicae Flos extract of the present invention, the increased amount of arachidonic acid due to UV irradiation was decreased to the level of non-exposure group. Therefore, it could be confirmed that the above result in Experimental Example 2-1 has no large error.

### Experimental Example 2-3

Further, to more accurately observe the change in the amount of PGE₂, an experiment identical with Experimental Example 2-1 was repeated using 500 μg/ml of Persicae Flos extract without isotope treatment, UV-B irradiation and 6-hour-incubation were conducted and the amount of PGE₂ was determined by ELISA.

Experimental result is shown in Table 2 and it could be confirmed that Persicae Flos extract according to the present invention inhibits the formation of PGE₂.

TABLE 2
Inhibition Effect against PGE₂ Formation (n = 3)

| Test group | PGE₂ (pg/mg protein) |
|---|---|
| Control group | 141 ± 3 |
| UV-irradiated group | 179 ± 7 |
| UV-irradiated and Persicae Flos extract | 122 ± 6* |
| (Ex. 4)-treated group | |

*Significant difference is recognized when compared to UV irradiation group (p < 0.01).

### Experimental Example 2-4

In addition, in order to confirm that the inhibition effect according to the present invention does not result from cell death, death rate of keratinocyte was assayed for the test groups in Experimental Example 2-1 (i.e. the groups treated before UV irradiation) according to MTT method [3-(4,5-dimethylthiazol-2-yl) 2,5-diphenyltetrazolin bromide].

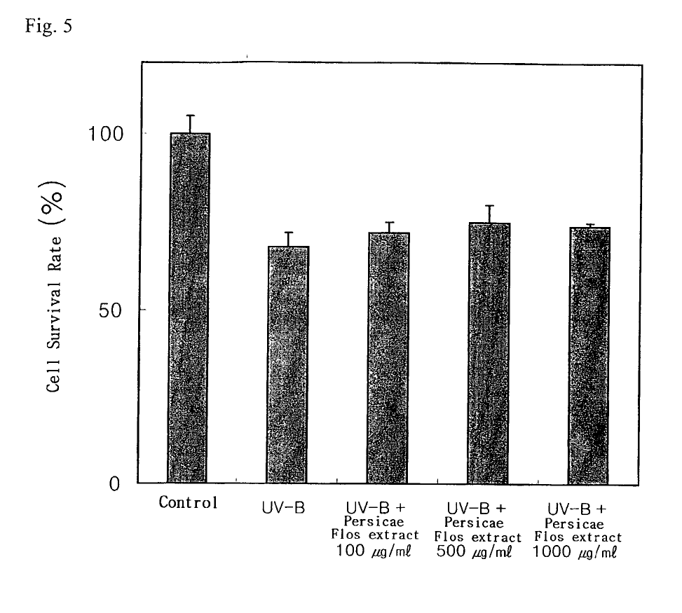

The result is shown in FIG. 5. It can be seen that a part of keratinocyte was destroyed by UV irradiation, yet no significant change according to treatment of Persicae Flos extract of the present invention could be observed, further the average seems to increase a little by the treatment of Persicae Flos extract in the present invention. Therefore, it could be confirmed that inhibition activity by Persicae Flos extract of the present invention, i.e., the results of Experimental Examples 2-1, 2 and 3, were not resulted from cell death.

### Experimental Example 3

### Destruction of Fibroblast by UV Irradiation

Like keratinocyte, Fibroblast is activated by UV irradiation. Furthermore, the cells of Fibroblast are destroyed as the amount of UV light increases, resulting in stimulation of skin aging and skin cancer due to DNA damage.

Therefore, in order to evaluate inhibitory effect of the Persicae Flos extract of the present invention on the destruction of fibroblast by UV irradiation, fibroblast was treated with the Persicae Flos extract of the present invention, and was exposed to U. Then its cell survival rate was measured according to MTT method.

Firstly, cell suspension was put in 96-well plate in an amount of 2.5×10⁴/well, and culture media was put and incubated for 24 hours. This was treated with the Persicae Flos extract of Example 4 for 12 hours, culture media was removed and UV was irradiated. New media was filled and after 2-hour-incubation, MTT reagent was added, culture media was removed after 4 hours, 200 μl of DMSO was added to dissolve formazan crystal, and absorbance at 570 nm was measured by microplate reader. The result is shown in Table 3. For control group, UV irradiation and the treatment with Persicae Flos extract of the present invention were not conducted.

TABLE 3
Inhibition Effect of Persicae Flos Extract against Cell Death by
UV Irradiation (n = 3)

Cell Survival Rate (%)

| Treatment (μg/ml) | UV-B (5 J/cm²) | UV-C (0.5 J/cm²) |
|---|---|---|
| 0 | 100 | 100 |
| 10 | 106.8 ± 5.3 | 146.8 ± 23.2 |
| 100 | 85.4 ± 2.9 | 154.2 ± 13.4* |

*P < 0.05 (Student t-test: Significant difference is recognized compared to group without treatment with the Persicae Flos extract)

It could be confirmed that the Persicae Flos extract of the present invention inhibits destruction of fibroblast by UV exposure. In particular, a remarkable effect was exhibited in UV-C region.

### Experimental Example 4

### Damage of Fibroblast DNA by UV Irradiation

UV irradiation damages fibroblast DNA. Thus, inhibition of this damage is a critical factor in prevention of skin cancer and retardation of skin aging. Therefore, to confirm the inhibitory effect of the Persicae Flos extract against this DNA damage, the following experiment was carried out.

Cells were put in a 24-well plate in an amount of 5×10⁴/well and then incubated as in Experimental Example 3. To this, UV was irradiated and after 2 hours, single cell electrophoresis (Comet assay) was conducted and DNA damage level was determined based on tail length (μm) by using image analyzer (Comet 3.1, Kinetic imaging, England). The result is as shown in Table 4. For control group, UV irradiation and extract treatment were not performed.

TABLE 4
Inhibitory effect of the Persicae Flos Extract against DNA
Damage by UV Irradiation

| | DNA damage (tail length: μm) | | | | | | | |
|---|---|---|---|---|---|---|---|---|
| | UV-B (5 J/cm²) | | | | UV-C (0.5 J/cm²) | | | |
| Treatment | 1st | 2nd | | Inhibition | 1st | 2nd | | Inhibition |
| (μg/ml) | treatment | treatment | Average | (%) | treatment | treatment | Average | (%) |
| Control | 47.1 | 44.6 | 45.8 | — | 48.7 | 33.4 | 41.0 | — |
| 0 | 152.8 | 155.0 | 153.9 | — | 160.7 | 177.9 | 169.3 | — |
| 50 | 178.5 | 146.2 | 162.3 | — | 164.8 | 116.2 | 140.5 | 17.0 |
| 100 | 107.5 | 145.0 | 126.2 | 17.9 | 113.5 | 138.4 | 125.9 | 25.6 |
| 150 | 91.5 | 108.7 | 100.1 | 34.9* | 117.0 | 118.4 | 117.7 | 30.4* |
| 200 | 124.5 | 122.4 | 123.4 | 19.8** | 131.2 | 107.9 | 119.5 | 29.4 |

*P < 0.05 (Student t-test: Significant difference is recognized compared to the group without extract treatment)
*P < 0.01 (Student t-test: Significant difference is recognized compared to the group without extract treatment)

It was confirmed that the Persicae Flos extract of the present invention inhibits DNA damage of fibroblast due to UV.

### Experimental Example 5

### Acute Toxicity

The Persicae Flos extract prepared in Example 4 was applied to skin of ICR mouse and of SD rat in an amount of 1 g/cm², and its side effects were observed.

As the result, neither side effect such as rube nor death of the subject was observed.

What is claimed is:

1. A composition for skin-protection against UV, comprising a Persicae Flos extract.

2. The composition in claim 1, wherein the content of the Persicae Flos extract is 0.01˜10% by weight.

3. The composition in claim 1, wherein the Persicae Flos extract is obtained by extracting a dried Persicae Flos with organic solvent or a mixture of organic solvent and water.

4. The composition in claim 1, which is applied to skin in an amount of 0.1-100 mg/cm2 based on Persicae Flos extract.
